(19) United States
(12) Patent Application Publication
(10) Pub. No.: US 2024/0189543 A1
(43) Pub. Date: Jun. 13, 2024

(54) INTRAVASCULAR LITHOTRIPSY CATHETER WITH RAPID EXCHANGE PORT

(71) Applicant: Bolt Medical, Inc., Carlsbad, CA (US)

(72) Inventors: Alvin Salinas, San Marcos, CA (US); Eric Schultheis, San Clemente, CA (US); Thanh Tran, Chula Vista, CA (US); Geraldine Paragas, San Marcos, CA (US)

(21) Appl. No.: 18/500,752

(22) Filed: Nov. 2, 2023

Related U.S. Application Data
(60) Provisional application No. 63/431,251, filed on Dec. 8, 2022.

(51) Int. Cl. A61M 25/00; A61B 18/26

(52) U.S. Cl. CPC A61M 25/0015; A61B 18/26; A61M 2025/0183

(57) ABSTRACT

A method for manufacturing a catheter (102) including a rapid exchange port (157). The method can include the steps of creating a port (258) in a catheter shaft (210), inserting a port tube (360) into the port (358), skiving the port tube (360) so that it is flush with the catheter shaft (310), inserting a guidewire lumen (718) into the port tube (760), coupling the guidewire lumen (718) to the port tube (760), and skiving the guidewire lumen (718) so that it is flush with the catheter shaft. The method can also include the steps of inserting a port mandrel (462) into the port tube (460), inserting a catheter mandrel (464) into the catheter shaft (410), positioning a heat shrink (465) over a portion of the catheter shaft (410), applying heat to the heat shrink (465), removing the port mandrel (462) from the port tube (460), removing the catheter mandrel from (464) the catheter shaft (410), and sealing a gap between the guidewire lumen (718) and the port tube (760).

### RELATED APPLICATION

This Application is related to and claims priority on U.S. Provisional patent application Ser. No. 63/431,251, filed on Dec. 8, 2022, and entitled “INTRAVASCULAR LITHOTRIPSY CATHETER WITH RAPID EXCHANGE PORT.” To the extent permissible, the contents of U.S. Provisional Application Ser. No. 63/431,251 are incorporated in their entirety herein by reference.

### BACKGROUND

Vascular lesions within vessels in the body can be associated with an increased risk for major adverse events, such as myocardial infarction, embolism, deep vein thrombosis, stroke, and the like. Severe vascular lesions, such as severely calcified vascular lesions, can be difficult to treat and achieve patency for a physician in a clinical setting.

Vascular lesions may be treated using interventions such as drug therapy, balloon angioplasty, atherectomy, stent placement, vascular graft bypass, to name a few. Such interventions may not always be ideal or may require subsequent treatment to address the lesion.

Intravascular lithotripsy is one method that has been recently used with some success for breaking up vascular lesions within vessels in the body. Intravascular lithotripsy utilizes a combination of pressure waves and bubble dynamics that are generated intravascularly in a fluid-filled balloon catheter. In particular, during an intravascular lithotripsy treatment, a high energy source is used to create plasma and, ultimately, pressure waves and a rapid bubble expansion within a fluid-filled balloon to crack calcification at a treatment site within the vasculature that includes one or more vascular lesions. The associated rapid bubble formation from the plasma initiation and resulting localized fluid velocity within the balloon transfers mechanical energy through the incompressible fluid to impart a fracture force on the intravascular calcium, which is opposed to the balloon wall. The rapid change in fluid momentum upon hitting the balloon wall is known as hydraulic shock, or water hammer.

There is an ongoing desire to enhance vessel patency and optimization of therapy delivery parameters within an intravascular lithotripsy catheter system in a manner that is relatively easy to control and is consistently manufacturable.

### SUMMARY

The present invention is directed toward a method for manufacturing a catheter including a rapid exchange port. In various embodiments, the method includes the steps of: puncturing a port in a catheter shaft; inserting a port tube into the port; skiving the port tube so that it is flush with the catheter shaft; inserting a guidewire lumen into the port tube; coupling the guidewire lumen to the port tube, and skiving the guidewire lumen so that it is flush with the catheter shaft.

In some embodiments, the method further comprises the step of inserting a port mandrel into the port tube.

In certain embodiments, the method further comprises the step of inserting a catheter mandrel into the catheter shaft.

In various embodiments, the method further comprises the step of positioning a heat shrink over a portion of the catheter shaft.

In some embodiments, the method further comprises the step of applying heat to the heat shrink.

In certain embodiments, the method further comprises the step of removing the port mandrel from the port tube.

In various embodiments, the method further comprises the step of removing the catheter mandrel from the catheter shaft.

In some embodiments, the method further comprises the step of sealing a gap between the guidewire lumen and the port tube.

In certain embodiments, the method further comprises the step of inserting an energy guide into the catheter shaft so that the energy guide is substantially parallel to the guidewire lumen.

In various embodiments, the energy guide is an optical fiber.

In some embodiments, the guidewire lumen is at least partially formed from a polymeric material.

In certain embodiments, the catheter shaft is at least partially formed from a polymeric material.

In various embodiments, the step of creating the port includes puncturing the catheter shaft.

In some embodiments, the mandrels are at least partially formed from a flexible material.

In certain embodiments, the mandrels are at least partially curved.

In various embodiments, the catheter mandrel is substantially u-shaped.

In some embodiments, the step of inserting the catheter mandrel into the catheter shaft includes positioning the port tube and the port mandrel in a curved portion of the catheter mandrel.

In certain embodiments, the heat shrink includes heat shrink tubing.

In various embodiments, the step of skiving the port tube is completed using a cutting tool.

In some embodiments, the step of skiving the guidewire lumen is completed using a cutting tool.

In certain embodiments, the step of sealing the gap is completed using an adhesive.

The present invention is also directed toward a catheter including a rapid exchange port. In certain embodiments, the catheter includes an energy guide, a catheter shaft, a port tube, and a guidewire lumen. The catheter shaft is configured to receive the energy guide. The catheter shaft can have (i) a shaft wall, and (ii) a port positioned on the shaft wall. The port tube can be positioned within the port. The port tube can have a tube end that is flush with the shaft wall. The guide wire lumen can be positioned within the port tube. The guidewire lumen can have a lumen end that is flush with the shaft wall and the tube end to form the rapid exchange port.

In various embodiments, the catheter can further include an adhesive that couples the guidewire lumen to the port tube.

In some embodiments, the adhesive at least partially encircles the guidewire lumen.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic cross-sectional view illustration of an embodiment of a catheter system in accordance with various embodiments;

FIG. 2 is a simplified cross-sectional of a portion of an embodiment of a catheter at an initial step in a method for manufacturing a catheter having a rapid exchange port;

FIG. 3 is a simplified, cross-sectional view of a portion of an embodiment of a catheter at a subsequent step in a method for manufacturing a catheter having a rapid exchange port;

FIG. 4 is a simplified, cross-sectional view of a portion of an embodiment of a catheter at a subsequent step in a method for manufacturing a catheter having a rapid exchange port;

FIG. 5 is a cross-sectional view of the embodiment of the catheter mandrel taken on lines 5-5 in FIG. 4;

FIG. 6 is a simplified, cross-sectional view of a portion of an embodiment of a catheter at a subsequent step in a method for manufacturing a catheter having a rapid exchange port;

FIG. 7 is a simplified, cross-sectional view of a portion of an embodiment of a catheter at a subsequent step in a method for manufacturing a catheter having a rapid exchange port;

FIG. 8A is a simplified, cross-sectional view of a portion of an embodiment of a catheter at a subsequent step in a method for manufacturing a catheter having a rapid exchange port

FIG. 8B is a simplified, cross-sectional view of a portion of the embodiment of a catheter having a rapid exchange port shown in 8A, with a guidewire inserted into the rapid exchange port; and

FIG. 9 is a flowchart outlining one embodiment of a method for manufacturing a catheter having a rapid exchange port.

While embodiments of the present invention are susceptible to various modifications and alternative forms, specifics thereof have been shown by way of examples and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a catheter configured to advance to a vascular lesion, such as a calcified vascular lesion or a fibrous vascular lesion, at a treatment site located within or adjacent to a blood vessel within a body of a patient. As used herein, the terms “treatment site,” “intravascular lesion,” and “vascular lesion” are used interchangeably unless otherwise noted. As such, the intravascular lesions and/or the vascular lesions are sometimes referred to herein simply as “lesions.”

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention, as illustrated in the accompanying drawings. The same or similar nomenclature and/or reference indicators will be used throughout the drawings, and the following detailed description to refer to the same or like parts.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is recognized that such a development effort might be complex and time-consuming. However, it would nevertheless be a routine engineering undertaking for those of ordinary skill in the art having the benefit of this disclosure.

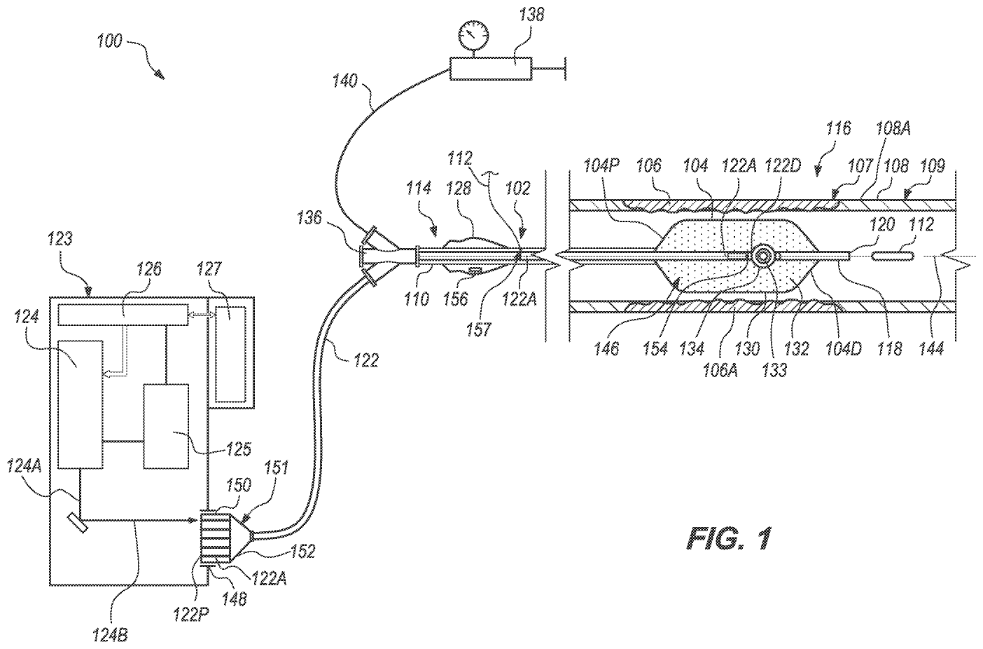

The catheter systems disclosed herein can include many different forms. Referring now to FIG. 1, a simplified schematic cross-sectional view illustration is shown of a catheter system 100 in accordance with various embodiments. The catheter system 100 is suitable for imparting pressure waves to induce fractures in one or more vascular lesions within or adjacent to a vessel wall of a blood vessel or on or adjacent to a heart valve within a body of a patient. In the embodiment illustrated in FIG. 1, the catheter system 100 can include one or more of a catheter 102, an energy guide bundle 122 including one or more energy guides 122A, a source manifold 136, a fluid pump 138, a system console 123 including one or more of an energy source 124, a power source 125, a system controller 126, and a graphic user interface 127 (a “GUI”), and a handle assembly 128. Alternatively, the catheter system 100 can include more components or fewer components than those specifically illustrated and described in relation to FIG. 1.

The catheter 102 is configured to move to the treatment site 106 within or adjacent to a vessel wall 108A of a blood vessel 108 within a body 107 of a patient 109. The treatment site 106 can include one or more vascular lesions 106A, such as calcified vascular lesions, for example. Additionally, or in the alternative, the treatment site 106 can include vascular lesions 106A, such as fibrous vascular lesions. Still alternatively, in some implementations, the catheter 102 can be used at a treatment site 106 within or adjacent to a heart valve within the body 107 of the patient 109.

The catheter 102 can include an inflatable balloon 104 (sometimes referred to herein simply as a “balloon”), a catheter shaft 110, and a guidewire 112. The balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter 102 and/or the catheter shaft 110 can also include a guidewire lumen 118, which is configured to move over the guidewire 112. As utilized herein, the guidewire lumen 118 defines a conduit through which the guidewire 112 extends. The catheter shaft 110 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106. In some embodiments, the balloon proximal end 104P can be coupled to the catheter shaft 110, and the balloon distal end 104D can be coupled to the guidewire lumen 118.

The balloon 104 includes a balloon wall 130 that defines a balloon interior 146. The balloon 104 can be selectively inflated with a catheter fluid 132 to expand from a deflated state suitable for advancing the catheter 102 through a patient's vasculature, to an inflated state (as shown in FIG. 1) suitable for anchoring the catheter 102 in position relative to the treatment site 106. Stated in another manner, when the balloon 104 is in the inflated state, the balloon wall 130 of the balloon 104 is configured to be positioned substantially adjacent to the treatment site 106. It is appreciated that although FIG. 1 illustrates the balloon wall 130 of the balloon 104 being shown spaced apart from the treatment site 106 of the blood vessel 108 when in the inflated state, this is done for ease of illustration. It is recognized that the balloon wall 130 of the balloon 104 will typically be substantially directly adjacent to and/or abutting the treatment site 106 when the balloon 104 is in the inflated state.

The balloon 104 suitable for use in the catheter system 100 includes those that can be passed through the vasculature of a patient 109 when in the deflated state. In some embodiments, the balloons 104 are made from silicone. In other embodiments, the balloon 104 can be made from materials such as polydimethylsiloxane (PDMS), polyurethane, polymers such as PEBAX™ material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In some embodiments, the balloon 104 can have a diameter (in the inflated state) ranging from at least two mm up to five mm.

In some embodiments, the balloon 104 can have a length ranging from at least three mm to 300 mm. More particularly, in some embodiments, the balloon 104 can have a length ranging from at least eight mm to 200 mm. It is appreciated that a balloon 104 having a relatively longer length can be positioned adjacent to larger treatment sites 106, and, thus, may be usable for imparting pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloon 104 can include a drug-eluting coating or a drug-eluting stent structure. The drug-eluting coating or drug-eluting stent can include one or more therapeutic agents, including anti-inflammatory agents, anti-neoplastic agents, anti-angiogenic agents, and the like.

The catheter fluid 132 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the pressure waves is appropriately manipulated. In certain embodiments, the catheter fluids 132 suitable for use are biocompatible. A volume of catheter fluid 132 can be tailored by the chosen energy source 124 and the type of catheter fluid 132 used.

In some embodiments, the contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine-based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C5F12).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG−emission maximum=1064 nm) lasers, holmium:YAG (Ho:YAG−emission maximum=2.1 μm) lasers, or erbium:YAG (Er:YAG−emission maximum=2.94 μm) lasers. In some embodiments, the absorptive agents can be water-soluble. In other embodiments, the absorptive agents are not water-soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the one or more energy guides 122A of the energy guide bundle 122, which are in optical communication with the energy source 124 via an optoelectrical connector assembly 151 (also referred to herein simply as an “optoelectrical connector”). Various embodiments of the optoelectrical connector 151 will be described in greater detail herein below.

The energy guide(s) 122A can be disposed along the catheter shaft 110 and within the balloon 104. In some embodiments, each energy guide 122A can be an optical fiber, and the energy source 124 can be a laser. The energy source 124 can be in optical communication with the energy guides 122A at the proximal portion 114 of the catheter system 100.

In some embodiments, the catheter shaft 110 can be coupled to multiple energy guides 122A, such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart by approximately 180 degrees about the circumference of the guidewire lumen 118 and/or the catheter shaft 110; three energy guides 122A can be spaced apart by approximately 120 degrees about the circumference of the guidewire lumen 118 and/or the catheter shaft 110; four energy guides 122A can be spaced apart by approximately 90 degrees about the circumference of the guidewire lumen 118 and/or the catheter shaft 110; six energy guides 122A can be spaced apart by approximately 60 degrees about the circumference of the guidewire lumen 118 and/or the catheter shaft 110; eight energy guides 122A can be spaced apart by approximately 45 degrees about the circumference of the guidewire lumen 118 and/or the catheter shaft 110, or ten energy guides 122A can be spaced apart by approximately 36 degrees about the circumference of the guidewire lumen 118 and/or the catheter shaft 110. Still, alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

The catheter system 100 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100 and/or the energy guide bundle 122 can include from one energy guide 122A to greater than 30 energy guides 122A. Alternatively, in other embodiments, the catheter system 100 and/or the energy guide bundle 122 can include greater than 30 energy guides 122A.

The energy guides 122A can have any suitable design for generating plasma and/or pressure waves in the catheter fluid 132 within the balloon interior 146. Thus, the general description of the energy guides 122A as light guides is not intended to be limiting in any manner except for as set forth in the claims appended hereto. More particularly, although the catheter systems 100 are often described with the energy source 124 as a light source and the one or more energy guides 122A as light guides, the catheter system 100 can alternatively include any suitable energy source 124 and energy guides 122A for purposes of generating the desired plasma in the catheter fluid 132 within the balloon interior 146. For example, in one non-exclusive alternative embodiment, the energy source 124 can be configured to provide high voltage pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the pressure waves in the catheter fluid 132 that are utilized to provide the fracture force onto the vascular lesions 106A at the treatment site 106. Still, alternatively, the energy source 124 and/or the energy guides 122A can have another suitable design and/or configuration.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P to the guide distal end 122D having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the length of the balloon 104 and/or relative to the length of the guidewire lumen 118 to more effectively and more precisely impart pressure waves for purposes of disrupting the vascular lesions 106A at the treatment site 106.

In certain embodiments, the energy guides 122A can include one or more photoacoustic transducers 154, where each photoacoustic transducer 154 can be in optical communication with the energy guide 122A within which it is disposed. In some embodiments, the photoacoustic transducers 154 can be in optical communication with the guide distal end 122D of the energy guide 122A. In such embodiments, the photoacoustic transducers 154 can have a shape that corresponds with and/or conforms to the guide distal end 122D of the energy guide 122A.

The photoacoustic transducer 154 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 154 disposed at the guide distal end 122D of the energy guide 122A can assume the same shape as the guide distal end 122D of the energy guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 154 disposed along one or more side surfaces of the length of the energy guide 122A.

In some embodiments, the energy guides 122A can further include one or more diverting features or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting feature can include any feature of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting feature. Stated in another manner, the diverting features can be configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting features suitable for use include a reflecting element, a refracting element, and a fiber diffuser. The diverting features suitable for focusing energy away from the tip of the energy guides 122A can include, but are not to be limited to, those having a convex surface, a gradient-index (GRIN) lens, and a mirror focus lens. Upon contact with the diverting feature, the energy is diverted within the energy guide 122A to one or more of a plasma generator 133 and the photoacoustic transducer 154 that is in optical communication with a side surface of the energy guide 122A. When utilized, the photoacoustic transducer 154 then converts light energy into an acoustic wave that extends away from the side surface of the energy guide 122A.

The source manifold 136 can be positioned at or near the proximal portion 114 of the catheter system 100. The source manifold 136 can include one or more proximal end openings that can receive the plurality of energy guides 122A of the energy guide bundle 122, the guidewire 112, and/or an inflation conduit 140 that is coupled in fluid communication with the fluid pump 138. Additionally, or in the alternative, in some embodiments, the source manifold 136 can be integrated and/or incorporated within the handle assembly 128.

The catheter system 100 can also include the fluid pump 138 that is configured to inflate the balloon 104 with the catheter fluid 132 as needed.

As noted above, in the embodiment illustrated in FIG. 1, the system console 123 includes one or more of the energy source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, and the GUI 127 can be provided at any suitable location within the catheter system 100 without the specific need for the system console 123.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket” or a “console receptacle”) by which the energy guide bundle 122 is mechanically coupled to the system console 123. In such embodiments, the energy guide bundle 122 can include the optoelectrical connector 151 having a guide coupling housing 150 (also sometimes referred to generally as a “connector housing”) that houses a portion, such as the guide proximal end 122P, of each of the energy guides 122A. At least a portion of the guide coupling housing 150 is configured to fit and be selectively retained within the console connection aperture 148 to provide the mechanical coupling between the energy guide bundle 122 and the system console 123.

The energy guide bundle 122 and/or the optoelectrical connector 151 can also include a guide bundler 152 (or “shell”) that provides strain relief as it brings each of the individual energy guides 122A closer together so that the energy guides 122A and/or the energy guide bundle 122 can be in a more compact form as it extends with the catheter 102 into the blood vessel 108 during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the properly aligned energy guides 122A in the energy guide bundle 122 as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104, such as via the plasma generator 133 that can be located at or near the guide distal end 122D of the energy guide 122A. In particular, in such embodiments, the energy emitted at the guide distal end 122D of the energy guide 122A is directed toward and energizes the plasma generator 133 to form the plasma in the catheter fluid 132 within the balloon interior 146. The plasma formation causes rapid bubble formation, and imparts pressure waves upon the treatment site 106. An exemplary plasma-induced bubble 134 is illustrated in FIG. 1.

In various non-exclusive alternative embodiments, the sub-millisecond pulses of energy from the energy source 124 can be delivered to the treatment site 106 at a frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a frequency that can be greater than 5000 Hz or less than one Hz, or any other suitable range of frequencies.

It is appreciated that although the energy source 124 is typically utilized to provide pulses of energy, the energy source 124 can still be described as providing a single source beam 124A, such as a single pulsed source beam.

The energy sources 124 suitable for use can include various types of light sources including lasers and lamps. Alternatively, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths, and energy levels that can be employed to achieve plasma in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 KHz.

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 in the catheter fluid 132.

The catheter system 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In various non-exclusive alternative embodiments, the catheter systems 100 can generate pressure waves having maximum pressures in the range of at least approximately two MPa to 50 MPa, at least approximately two MPa to 30 MPa, or approximately at least 15 MPa to 25 MPa.

The pressure waves can be imparted upon the treatment site 106 from a distance within a range from at least approximately 0.1 millimeters (mm) to greater than approximately 25 mm, extending radially from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to 30 MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

The power source 125 is electrically coupled to and is configured to provide the necessary power to each of the energy source 124, the system controller 126, the GUI 127, and the handle assembly 128. The power source 125 can have any suitable design for such purposes.

The system controller 126 is electrically coupled to and receives power from the power source 125. The system controller 126 is coupled to and is configured to control the operation of each of the energy source 124 and the GUI 127. The system controller 126 can include one or more processors or circuits for purposes of controlling the operation of at least the energy source 124 and the GUI 127. For example, the system controller 126 can control the energy source 124 for generating pulses of energy as desired and/or at any desired firing rate.

The system controller 126 can also be configured to control the operation of other components of the catheter system 100, such as the positioning of the catheter 102 adjacent to the treatment site 106, the inflation of the balloon 104 with the catheter fluid 132, etc. Further, or in the alternative, the catheter system 100 can include one or more additional controllers that can be positioned in any suitable manner to control the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. The GUI 127 is electrically connected to the system controller 126. With such design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during, and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during the use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications, and/or desires of the user or operator.

As shown in FIG. 1, the handle assembly 128 can be positioned at or near the proximal portion 114 of the catheter system 100. In this embodiment, the handle assembly 128 is coupled to the balloon 104 and positioned separately from the balloon 104. Alternatively, the handle assembly 128 can be positioned at another suitable location.

The handle assembly 128 is attached to the catheter shaft 110 and is handled and used by the user or operator to operate, position, and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, and the GUI 127.

In some embodiments, the handle assembly 128 can integrate and/or include at least a portion of the system controller 126 within an interior of the handle assembly 128. For example, as shown, in certain such embodiments, the handle assembly 128 can include circuitry 156, which is electrically coupled between catheter electronics and the system console 123, and which can form at least a portion of the system controller 126. In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, such as within the system console 123. It is understood that the handle assembly 128 can include fewer or additional components than those specifically illustrated and described herein.

The catheter system 100 can also include the fluid pump 138 that is configured to inflate the balloon 104 with the catheter fluid 132 as needed.

In various embodiments, the catheter 102 can include a rapid exchange port 157 that is positioned on a portion of the catheter shaft 110. The rapid exchange port 157 can be configured to receive the guidewire 112 (and any other suitable component of the catheter system 100) at a position that is distal to the handle assembly 128. In other embodiments, the rapid exchange port 157 can be positioned proximal to the handle assembly 128.

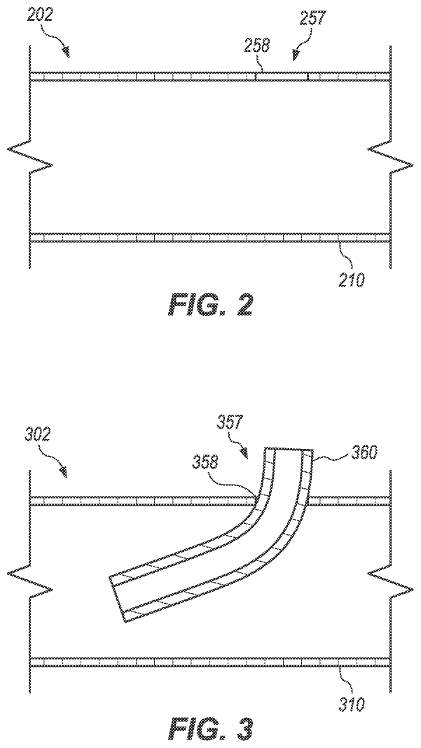

The design of the rapid exchange port 157 can vary depending on the design requirements of the catheter system 100 and the catheter 102. In certain embodiments, the rapid exchange port 157 can include one or more of (i) a port 258 (for example, as illustrated in FIG. 2), (ii) a port tube 360 (for example, as illustrated in FIG. 3), and/or (iii) the guidewire lumen 118.

As used herein, the term “rapid exchange” should be understood as imparting the same or similar meanings as other terms used in the catheter arts, such as single operator exchange. In particular, a “rapid exchange catheter” includes any catheter designed to enable exchange of a catheter placed using a guidewire without requiring a guidewire extension or guidewire that is substantially longer than the catheter itself. While much of the following description and several of the figures illustrate balloon catheters, other catheter types may incorporate the improvements herein, including, for example, fluid infusion cannulas, cutting instruments, non-balloon self-expanding stent delivery catheters, and/or atherectomy devices. Vascular and non-vascular (such as biliary or uretogenital) uses are contemplated.

As with all embodiments illustrated and described herein, various structures may be omitted from the figures for clarity and ease of understanding. Further, the figures may include certain structures that can be omitted without deviating from the intent and scope of the invention.

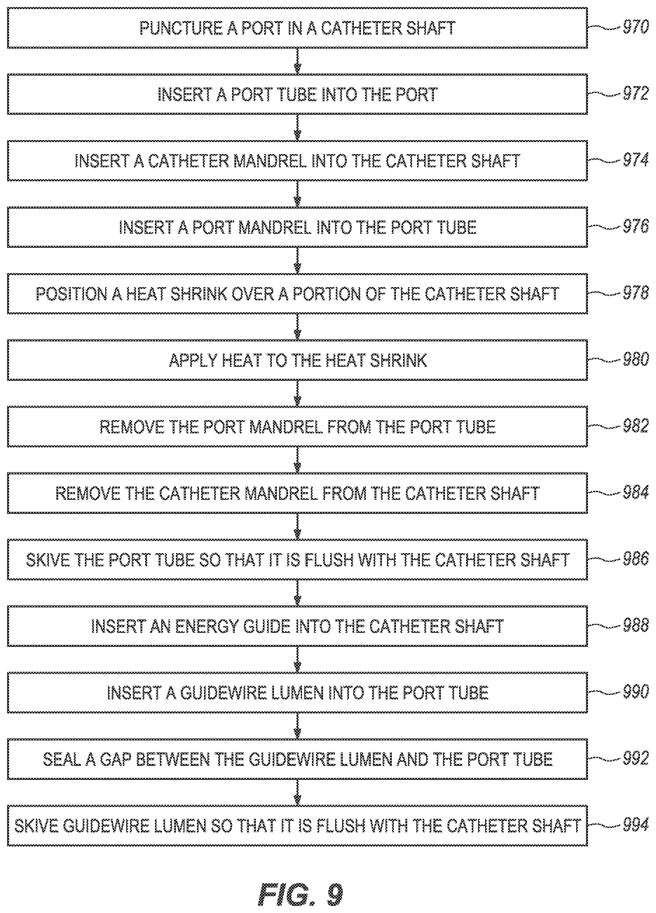

FIG. 2 is a simplified cross-sectional view of a portion of an embodiment of a catheter 202 at an initial step in a method for manufacturing a catheter 202 having a rapid exchange port 257. For example, the catheter 202 can have features of the present invention that can be included as part of a method for manufacturing a catheter 202 having a rapid exchange port 257, shown in FIG. 9.

The design of the catheter 202 can be varied. As shown, FIG. 2 illustrates an embodiment of the catheter 202, including the catheter shaft 210 and the rapid exchange port 257. The rapid exchange port 257 illustrated in FIG. 2 includes a port 258.

The port 258 can be configured to enable the rapid exchange of guidewire lumens 118 (for example, illustrated in FIG. 1) near the distal portion 116 (illustrated in FIG. 1) of the catheter 202. The design of the port 258 can be varied. The port 258 can be formed in catheter shaft 210 using any suitable method in the art. In some embodiments, the port 258 is formed in the catheter shaft 210 by puncturing, boring, piercing, and/or penetrating the catheter shaft 210 with a suitable tool.

FIG. 3 is a simplified, cross-sectional view of a portion of an embodiment of a catheter 302 at a subsequent step in a method for manufacturing a catheter 302 having a rapid exchange port 357. As shown in FIG. 3, in various embodiments, a port tube 360 can be positioned within the catheter shaft 310 via the port 358 to form the rapid exchange port 357. The embodiment of the catheter shaft 310 of the catheter 302, shown in FIG. 3, can be included as part of a method for manufacturing a catheter 302 having a rapid exchange port 357, shown in FIG. 9.

The port tube 360 can be configured to enable the rapid exchange of guidewire lumens 118 (for example, illustrated in FIG. 1) near the distal portion 116 (illustrated in FIG. 1) of the catheter 302. The design of the port tube 360 can be varied. The port tube 360 can include any suitable tube or conduit capable of receiving the guidewire lumen 118.

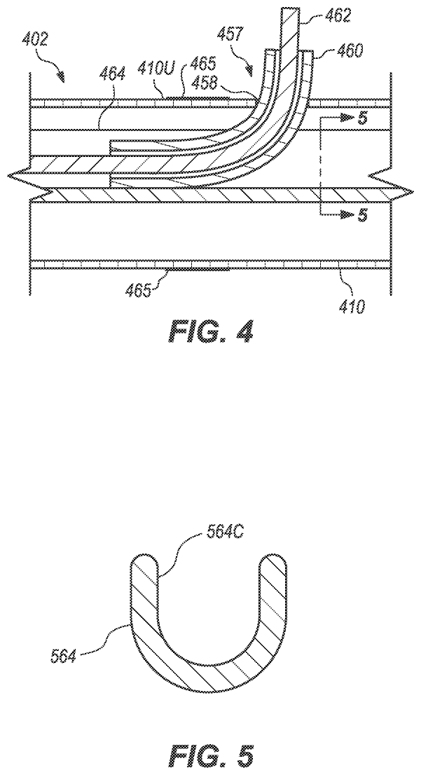

FIG. 4 is a simplified, cross-sectional view of a portion of an embodiment of a catheter 402 at a subsequent step in a method for manufacturing a catheter 402 having a rapid exchange port 457. As shown in FIG. 4, in various embodiments, the port tube 460 can be positioned within the catheter shaft 410 via the port 458 to form the rapid exchange port 457. The embodiment of the catheter shaft 410 of the catheter 402 shown in FIG. 4, can be included as part of a method for manufacturing a catheter 402 having a rapid exchange port 457, shown in FIG. 9. The catheter 402 can include a port mandrel 462 and a catheter mandrel 464.

The port mandrel 462 can support the port tube 460 during the method for manufacturing a rapid exchange catheter shown in FIG. 9. The port mandrel 462 can be positioned within the port tube 460 so that the port tube 460 is positioned in an upper portion of the catheter shaft 410, as shown in FIG. 4.

In certain embodiments, the port mandrel 462 can position the port tube 460 so that it is in contact with an upper shaft wall 410U of the catheter shaft 410. In other embodiments, the port mandrel 462 can position the port tube 460 so that the portion of the port tube 460 that is positioned within the catheter shaft 410 is substantially parallel to the upper shaft wall 410U of the catheter shaft 410.

The port mandrel 462 can vary depending on the design requirements of the catheter 402 and the catheter shaft 410. The port mandrel 462 can be at least partially formed from a flexible material. The port mandrel 462 can be at least partially curved.

The catheter mandrel 464 can support the port tube 460, and the port mandrel 462, during the method for manufacturing a catheter 402, having a rapid exchange port 457, shown in FIG. 9. The catheter mandrel 464 can be positioned within the catheter shaft 410 so that the port tube 460 and the port mandrel 462 are positioned in a curved portion of the catheter mandrel 464, as shown in FIG. 4.

In some embodiments, the catheter mandrel 464 can position the port tube 460 and the port mandrel 462 so that the port tube 460 is in contact with the upper shaft wall 410U of the catheter shaft 410. In other embodiments, the catheter mandrel 464 can position the port tube 460 so that the portion of the port tube 460 that is positioned within the catheter shaft 410 is substantially parallel to the upper shaft wall 410U of the catheter shaft 410.

The catheter mandrel 464 can vary depending on the design requirements of the catheter 402, the catheter shaft 410, the port tube 460, and/or the port mandrel 462. The port mandrel 462 can be at least partially formed from a flexible material. The port mandrel 462 can be at least partially curved.

A heat shrink 465 can be positioned over a portion of the catheter shaft 410. Non-limiting, non-exclusive example of suitable heat shrinks 465 include heat shrink tubing, heat shrink wrap, and/or heat shrink sleeves. In certain embodiments, the heat shrink 465 can include a heat sink tube that is slid over a portion of the catheter shaft 410 that contains the port 458, the port tube 460, the port mandrel 462, and/or the catheter mandrel 464. Heat can be applied to the heat shrink 465, and the contents within the heat shrink 465. The heat shrink 465, and the insulated contents within can be shrunk upon the application of heat.

FIG. 5 is a cross-sectional view of an embodiment of a catheter mandrel 564 taken on line 5-5 in FIG. 4. In the embodiment illustrated in FIG. 5, the catheter mandrel 564 can be substantially U-shaped and can include a mandrel curved surface 564C. As shown in FIG. 4, the port tube 460 (illustrated in FIG. 4) and the port mandrel 462 (illustrated in FIG. 4) can be positioned within the mandrel curved surface 564C of the catheter mandrel 564 during the manufacturing of the catheter 402 (shown in FIG. 4) and/or the catheter shaft 410 (illustrated in FIG. 4). Alternatively, the mandrel 564 can have a different configuration, and/or can include a different mandrel curved surface 564C than that illustrated in FIG. 5. In one non-exclusive embodiment, the mandrel 564 can include a metal rod and/or a metal tube, such as an SS Hypotube, for example. Any suitable materials can be used.

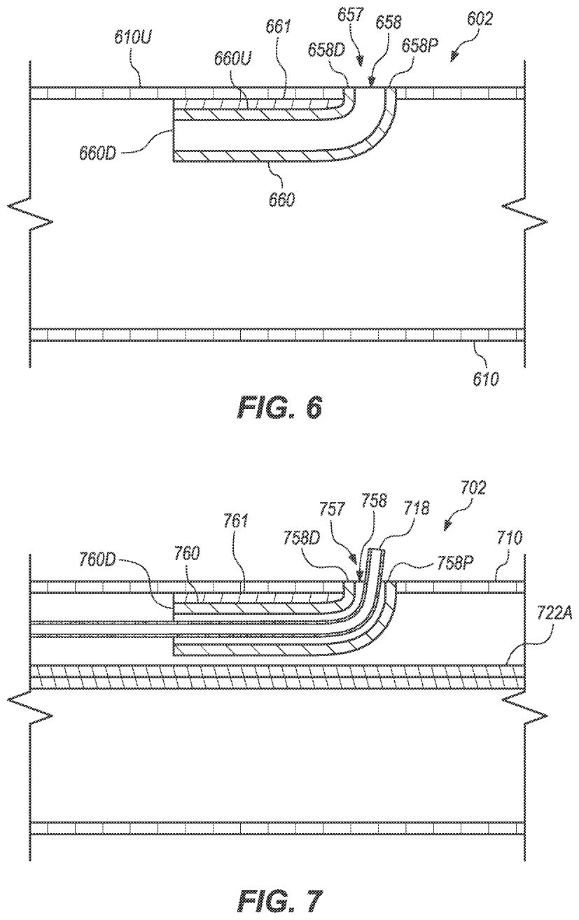

FIG. 6 is a simplified, cross-sectional view of an embodiment of a catheter shaft 610 of the catheter 602. As shown in FIG. 6, the port 658 of the rapid exchange port 657 can include a port distal shoulder 658D and a port proximal shoulder 658P. The embodiment of the catheter shaft 610 of the catheter 602 shown in FIG. 6 can be included as part of a method for manufacturing a catheter having a rapid exchange port shown in FIG. 9.

The port distal shoulder 658D is formed on the distal side of the port 658, where the distal portion of the port tube 660 meets the upper shaft wall 610U. The port distal shoulder 658D can be skived so that it is flush with both the upper shaft wall 610U and the port tube 660.

The port proximal shoulder 658P is formed on the proximal side of the port 658 where the proximal portion of the port tube 660 meets the upper shaft wall 610U. The port proximal shoulder 658P can be skived so that it is flush with both the upper shaft wall 610U and the port tube 660.

The port tube 660 can include a tube distal end 660D and a tube upper surface 660U. The tube distal end 660D can extend toward the distal portion 116 (for example, as illustrated in FIG. 1). The tube upper surface 660U can be substantially parallel to the catheter upper surface 610U. In some embodiments, the tube upper surface 660U can be fused to the upper shaft wall 610U to form a fused portion 661.

The fused portion 661 can include portions of both the catheter shaft 610 and the port tube 660 that are fused and/or reflowed together during the heating and/or reflow process that is described with respect to step 980 illustrated in FIG. 9. As shown in FIG. 6, the fused portion 660 can be formed directly between the port tube 660 and the catheter shaft 610. The fused portion 660 can anchor the position of the port tube 660 with respect to the catheter shaft 610.

FIG. 7 is a simplified, cross-sectional view of an embodiment of a catheter shaft 710 of the catheter 702. The embodiment of the catheter shaft 710 of the catheter 702 including the rapid exchange port 757 that is shown in FIG. 7 can be included as part of a method for manufacturing a catheter having a rapid exchange port shown in FIG. 9. In the embodiment illustrated in FIG. 7, the guidewire lumen 718 can be positioned in the rapid exchange port 757 through the port 758 between the port distal shoulder 758D and the port proximal shoulder 758P. The guidewire lumen 718 can extend through the port tube 760 distally out of the tube distal end 760D and toward the distal portion 116 (for example, as illustrated in FIG. 1). The guidewire lumen 718 can be substantially parallel to both the port tube 760 and the fused portion 761.

Energy guides 722A can extend through the catheter shaft 710 toward the distal portion 116. The energy guides 722A can be adjacent to the guidewire lumen 718 and the port tube 760 in the interior of the catheter shaft 710.

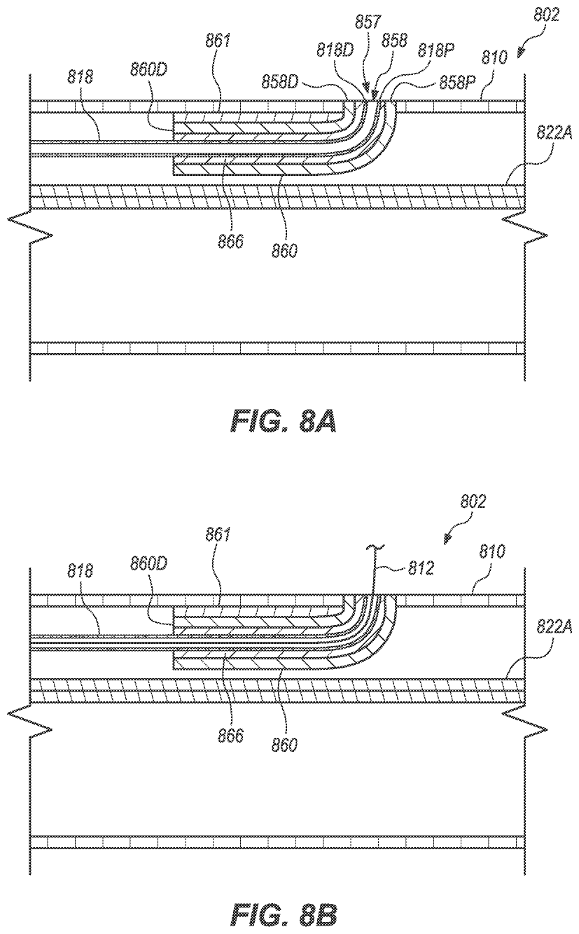

FIG. 8A is a simplified, cross-sectional view of an embodiment of a catheter shaft 810 of the catheter 802 including a rapid exchange port 857. The embodiment of the catheter shaft 810 of the catheter 802 shown in FIG. 8 can be included as part of a method for manufacturing a catheter having a rapid exchange port shown in FIG. 9. As shown in FIG. 8, an adhesive 866 can be applied to the interior of the port tube 860.

The adhesive 866 can couple the guidewire lumen 818 to the catheter shaft 810 and the port tube 860. As shown in FIG. 8, the guidewire lumen 818 and the adhesive 866 can be skived so that they are flush with the catheter shaft 810. The adhesive 866 can seal the gaps between (i) the tube distal end 860D, the port distal shoulder 858D, a guidewire distal shoulder 818D, and (ii) the tube distal end 860D, a guidewire proximal shoulder 818P, and the port proximal shoulder 858P. The guidewire lumen 818 can be substantially adjacent to the energy guides 822A. In some embodiments, such as shown in FIG. 8, an upper portion of the rapid exchange port 857 can be formed so that a portion of the catheter shaft 810, the fused portion 861, a portion of the port tube 860, a portion of the adhesive 866, a portion of the guidewire lumen 818 are coupled together in layers.

FIG. 8B is a simplified, cross-sectional view of a portion of the embodiment of the catheter 802 having a rapid exchange port 857 shown in 8A, with a guidewire 812 inserted into the rapid exchange port 857. As shown in FIG. 8B, the guidewire 812 can be inserted and removed from the guidewire lumen 818 via the rapid exchange port 857.

FIG. 9 is a flowchart outlining one embodiment of a method for manufacturing a catheter having a rapid exchange port. The method can include one or more of the following steps provided herein. It is understood that the method can include additional steps other than those specifically shown and/or described herein. Additionally, or alternatively, the method can omit one or more of the steps that are specifically shown and/or described herein. Further, it is understood that the steps can be completed in any order, and the order of steps shown and/or described herein is merely for illustrative purposes. It is also appreciated that any of the steps shown and/or described herein can be combined and completed in a single step, and/or a single step can be spread out over multiple steps.

The method for manufacturing the catheter including a rapid exchange port can include manufacturing catheters that can be utilized in the catheter system 100 (illustrated in FIG. 1) or other suitable systems and subsystems not explicitly shown and/or described herein.

At step 970, a port is punctured in a catheter shaft. In some embodiments, the port can be formed in the catheter shaft via any suitable port formation method. Non-limiting, non-exclusive examples include boring, puncturing, piercing, perforating, gouging, and cutting. It is appreciated that in certain embodiments, the tool used to form the port in the catheter shaft should be configured to pierce a catheter shaft of any suitable material, including polymeric materials.

At step 972, a port tube is inserted into the port. The port tube can be inserted so that a portion of the port tube extends into the interior of the catheter shaft and a portion of the port tube extends externally from the catheter shaft.

At step 974, a catheter mandrel is inserted into the catheter shaft. The catheter mandrel can include flexible materials. The catheter mandrel can be inserted so that a portion of the catheter mandrel extends into the interior of the catheter shaft and a portion of the catheter mandrel extends externally from the catheter shaft. The catheter mandrel can have a curved portion. The curved portion of the catheter mandrel can receive and/or house the port tube and the port mandrel. The catheter mandrel can position the port tube and the port mandrel in any suitable position within the interior of the catheter shaft.

At step 976, a port mandrel is inserted into the port tube. The port mandrel can include flexible materials. The port mandrel can be inserted so that a portion of the port mandrel extends into the interior of (i) the catheter shaft, and (ii) the port tube, and a portion of the port mandrel extends externally from (i) the catheter shaft, and (ii) the port tube.

At step 978, a heat shrink is positioned over a portion of the catheter shaft. Non-limiting, non-exclusive examples of suitable heat shrinks include heat shrink tubing, heat shrink wrap, and/or heat shrink sleeves. In certain embodiments, the heat shrink can include a heat sink tube that is slid over a portion of the catheter shaft that contains the port, port tube, port mandrel, and catheter mandrel.

At step 980, heat is applied to the heat shrink, and the contents within the heat shrink. The port mandrel can prevent the port tube from collapsing during heating, and the catheter mandrel can prevent the catheter shaft from collapsing during heating. The port mandrel can maintain the position of the port tube during heating, and the catheter mandrel maintain the position of the catheter shaft during heating. The heat reflows the catheter shaft and the port tube together. In certain embodiments, the heating creates a fused portion where portions of the catheter shaft and port tube are fused together.

At step 982, the port mandrel is removed from the port tube.

At step 984, the catheter mandrel is removed from the catheter shaft.

At step 986, the port tube is skived so that it is flush with the catheter shaft. Portions of the port tube are skived so that the size of the port is maintained, and the port tube is contiguous with the catheter shaft. The port tube can be skived and/or cut using any suitable method. In some embodiments, the port tube is skived with a cutting tool. Non-limiting, not-exclusive examples of cutting tools include razors, knives, and/or rotary cutters.

At step 988, an energy guide is fed into the catheter shaft. The energy guide can be placed in a position that is adjacent to the port tube and the guidewire lumen.

At step 990, a guidewire lumen is inserted into the port tube. The guidewire lumen can be inserted so that a portion of the guidewire lumen extends into the interior of (i) the catheter shaft, and (ii) the port tube, and a portion of the guidewire lumen extends externally from (i) the catheter shaft, and (ii) the port tube. The guidewire lumen can be positioned so that it extends from the port toward a distal portion of the catheter shaft.

At step 992, a gap is sealed between the guidewire lumen and the port tube. The gap can be sealed with an adhesive. The adhesive can substantially surround the portion of the guidewire lumen that is positioned within the port tube. Non-limiting, non-exclusive examples of suitable adhesives include glues, wicking glues, and/or sealants.

At step 994, the guidewire lumen is skived so that it is flush with the catheter shaft. Portions of the guidewire lumen are skived so that the size of the port matches the opening of the guidewire lumen, and the guidewire lumen is contiguous with the catheter shaft. The guidewire lumen can be skived and/or cut using any suitable method. In some embodiments, the guidewire lumen is skived with a cutting tool. Non-limiting, not-exclusive examples of cutting tools include razors, knives, and/or rotary cutters. The adhesive that is positioned between the guidewire lumen and the port tube can also be skived so that it is flush with the catheter shaft, the port tube, and the guidewire lumen.

In one non-exclusive embodiment, a method for manufacturing a catheter having a rapid exchange port can include the steps of puncturing a port in a catheter shaft; inserting a port tube into the port; skiving the port tube so that it is flush with the catheter shaft; inserting a guidewire lumen into the port tube; coupling (or otherwise securing) the guidewire lumen to the port tube; and skiving the guidewire lumen so that it is flush with the catheter shaft. It is understood that the foregoing example of one embodiment may also include additional steps, such as those disclosed herein, or may omit certain steps as needed.

The present technology is also directed toward methods for treating a treatment site within or adjacent to a vessel wall, with such methods utilizing the devices disclosed herein.

In summary, based on the various embodiments of the present invention illustrated and described in detail herein, the catheter systems and related methods can include a catheter configured to advance to a vascular lesion, such as a calcified vascular lesion, or a fibrous vascular lesion, at a treatment site located within or adjacent a blood vessel within a body of a patient. The catheter includes a catheter shaft, and an inflatable balloon that is coupled and/or secured to the catheter shaft. The balloon can include a balloon wall that defines a balloon interior. The balloon can be configured to receive a catheter fluid within the balloon interior to expand from a deflated state suitable for advancing the catheter through a patient's vasculature, to an inflated state suitable for anchoring the catheter in position relative to the treatment site.

In certain embodiments, the catheter systems and related methods utilize an energy source, e.g., a light source such as a laser source or another suitable energy source, which provides energy that is guided by one or more energy guides, e.g., light guides such as optical fibers, which are disposed along the catheter shaft and within the balloon interior of the balloon to create a localized plasma in the catheter fluid that is retained within the balloon interior of the balloon. The energy guide can be used in conjunction with a plasma generator that is positioned at or near a guide distal end of the energy guide within the balloon interior of the balloon located at the treatment site. The creation of the localized plasma can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles (also sometimes referred to simply as “plasma bubbles”) can generate one or more pressure waves in the catheter fluid retained within the balloon interior of the balloon and thereby impart pressure waves onto and induce fractures in the vascular lesions at the treatment site within or adjacent to the blood vessel wall within the body of the patient. In some embodiments, the energy source can be configured to provide sub-millisecond pulses of energy, e.g., light energy, to initiate the plasma formation in the catheter fluid within the balloon to cause the rapid bubble formation and to impart the pressure waves upon the balloon wall at the treatment site. Thus, the pressure waves can transfer mechanical energy through an incompressible catheter fluid to the treatment site to impart a fracture force on the intravascular lesion. Without wishing to be bound by any particular theory, it is believed that the rapid change in catheter fluid momentum upon the balloon wall that is in contact with the intravascular lesion is transferred to the intravascular lesion to induce fractures to the lesion.

It should be noted that, as used in this specification and the appended claims, the singular forms “a,” “an,” and “the” include plural referents unless the content and/or context clearly dictates otherwise. It should also be noted that the term “or” is generally employed in its sense, including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

It is recognized that the figures shown and described are not necessarily drawn to scale, and that they are provided for ease of reference and understanding, and for relative positioning of the structures.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. A method for manufacturing a catheter including a rapid exchange port, the method comprising the steps of:
puncturing a port in a catheter shaft;
inserting a port tube into the port;
skiving the port tube so that it is flush with the catheter shaft;
inserting a guidewire lumen into the port tube;
coupling the guidewire lumen to the port tube; and
skiving the guidewire lumen so that it is flush with the catheter shaft.

2. The method of claim 1 further comprising the step of inserting a port mandrel into the port tube.

3. The method of claim 2 wherein the port mandrel is at least partially formed from a flexible material.

4. The method of claim 2 wherein the port mandrel is at least partially curved.

5. The method of claim 2 further comprising the step of removing the port mandrel from the port tube.

6. The method of claim 1 further comprising the step of inserting a catheter mandrel into the catheter shaft.

7. The method of claim 6 wherein the step of inserting the catheter mandrel into the catheter shaft includes positioning the port tube and the port mandrel in a curved portion of the catheter mandrel.

8. The method of claim 6 further comprising the step of removing the catheter mandrel from the catheter shaft.

9. The method of claim 6 wherein the catheter mandrel is substantially u-shaped.

10. The method of claim 1 further comprising the step of positioning a heat shrink over a portion of the catheter shaft.

11. The method of claim 10 wherein the heat shrink includes heat shrink tubing.

12. The method of claim 10 further comprising the step of applying heat to the heat shrink.

13. The method of claim 1 further comprising the step of sealing a gap between the guidewire lumen and the port tube.

14. The method of claim 13 wherein the step of sealing the gap is completed using an adhesive.

15. The method of claim 1 further comprising the step of inserting an energy guide into the catheter shaft so that the energy guide is substantially parallel to the guidewire lumen.

16. The method of claim 15 wherein the energy guide is an optical fiber.

17. The method of claim 1 wherein at least one of the guidewire lumen and the catheter shaft is at least partially formed from a polymeric material.

18. The method of claim 1 wherein the step of skiving the port tube is completed using a cutting tool.

19. The method of claim 1 wherein the step of skiving the guidewire lumen is completed using a cutting tool.

20. A catheter including a rapid exchange port, the catheter comprising:
an energy guide; and
a catheter shaft that is configured to receive the energy guide, the catheter shaft having (i) a shaft wall, and (ii) a port positioned on the shaft wall;
a port tube that is positioned within the port, the port tube having a tube end that is flush with the shaft wall;
a guide wire lumen that is positioned within the port tube, the guidewire lumen having a lumen end that is flush with the shaft wall and the tube end to form the rapid exchange port; and
an adhesive that couples the guidewire lumen to the port tube.
